SEQUENCE LISTING (34 sequences)

SEQ ID NO: 1 - DNA, 24 bp, Artificial Sequence
  Synthetic (primer ACACA- F)
ttaacagctgtggagtctggctgt

SEQ ID NO: 2 - DNA, 24 bp, Artificial Sequence
  Synthetic (primer ACACA-R)
aacactcgatggagtttctcgcct

SEQ ID NO: 3 - DNA, 20 bp, Artificial Sequence
  Synthetic (primer Acot1- F)
tctttcctgaagggcatcac

SEQ ID NO: 4 - DNA, 20 bp, Artificial Sequence
  Synthetic (primer Acot1- R)
atgatctggggctttctcct

SEQ ID NO: 5 - DNA, 17 bp, Artificial Sequence
  Synthetic (primer B-actin-F)
gtggggcgccccaggca

SEQ ID NO: 6 - DNA, 21 bp, Artificial Sequence
  Synthetic (primer B-actin-R)
ctccttaatgtcacgcacgat

SEQ ID NO: 7 - DNA, 20 bp, Artificial Sequence
  Synthetic (primer E-cadherin-F)
tgcccagaaaatgaaaaagg

SEQ ID NO: 8 - DNA, 20 bp, Artificial Sequence
  Synthetic (primer E-cadherin-R)
gtgtatgtggcaatgcgttc

SEQ ID NO: 9 - DNA, 20 bp, Artificial Sequence
  Synthetic (primer ELOVL2-F)
ctctccacttgggaaggagg

SEQ ID NO: 10 - DNA, 20 bp, Artificial Sequence
  Synthetic (primer ELOVL2-R)
agatgagacaaccgaagggg

SEQ ID NO: 11 - DNA, 25 bp, Artificial Sequence
  Synthetic (primer ELOVL3-F)
ggtccttctgccttgcaatcttcag

SEQ ID NO: 12 - DNA, 28 bp, Artificial Sequence
  Synthetic (primer ELOVL3-R)
ttcactggctcttggtcttggctttgac

SEQ ID NO: 13 - DNA, 20 bp, Artificial Sequence
  Synthetic (primer EpCAM-F)
gcgttcgggcttctgcttgc

SEQ ID NO: 14 - DNA, 21 bp, Artificial Sequence
  Synthetic (primer EpCAM-R)
ccgctctcatcgcagtcagga

SEQ ID NO: 15 - DNA, 20 bp, Artificial Sequence
  Synthetic (primer FASN-F)
ggtcttgagagatggcttgc

SEQ ID NO: 16 - DNA, 20 bp, Artificial Sequence
  Synthetic (primer FASN-R)
cagtgagctgtccaggttga

SEQ ID NO: 17 - DNA, 20 bp, Artificial Sequence
  Synthetic (primer Fibronectin-F)
cagtgggagacctcgagaag

SEQ ID NO: 18 - DNA, 20 bp, Artificial Sequence
  Synthetic (primer Fibronectin-R)
tccctcggaacatcagaaac

SEQ ID NO: 19 - DNA, 20 bp, Artificial Sequence
  Synthetic (primer N-cadherin-F)
acagtggccacctacaaagg

SEQ ID NO: 20 - DNA, 20 bp, Artificial Sequence
  Synthetic (primer N-cadherin-R)
ccgagatggggttgataatg

SEQ ID NO: 21 - DNA, 20 bp, Artificial Sequence
  Synthetic (primer PECR-F)
atggaggaggccagtttctt

SEQ ID NO: 22 - DNA, 20 bp, Artificial Sequence
  Synthetic (primer PECR-R)
aggaagcagaccacagagga

SEQ ID NO: 23 - DNA, 20 bp, Artificial Sequence
  Synthetic (primer PPAR-gamma-F)
tctggcccaccaactttggg

SEQ ID NO: 24 - DNA, 20 bp, Artificial Sequence
  Synthetic (primer PPAR-gamma-R)
cttcacaagcatgaactcca

SEQ ID NO: 25 - DNA, 20 bp, Artificial Sequence
  Synthetic (primer PTPLB-F)
gtccgagcatacctggctaa

SEQ ID NO: 26 - DNA, 20 bp, Artificial Sequence
  Synthetic (primer PTPLB-R)
actcccattgggtacagcac

SEQ ID NO: 27 - DNA, 20 bp, Artificial Sequence
  Synthetic (primer SCD1-F)
ttggagaagcggtggataac

SEQ ID NO: 28 - DNA, 20 bp, Artificial Sequence
  Synthetic (primer SCD1-R)
aaaaatcccacccaatcaca

SEQ ID NO: 29 - DNA, 20 bp, Artificial Sequence
  Synthetic (primer Snail-F)
cctccctgtcagatgaggac

SEQ ID NO: 30 - DNA, 20 bp, Artificial Sequence
  Synthetic (primer Snail-R)
ccaggctgaggtattccttg

SEQ ID NO: 31 - DNA, 20 bp, Artificial Sequence
  Synthetic (primer Twist-F)
ggagtccgcagtcttacgag

SEQ ID NO: 32 - DNA, 20 bp, Artificial Sequence
  Synthetic (primer Twist-R)
tctggaggacctggtagagg

SEQ ID NO: 33 - DNA, 20 bp, Artificial Sequence
  Synthetic (primer Vimentin-F)
gagaactttgccgttgaaga

SEQ ID NO: 34 - DNA, 20 bp, Artificial Sequence
  Synthetic (primer Vimentin-R)
gcttcctgtaggtggcaatc

(19) United States
(12) Patent Application Publication
(10) Pub. No.: US 2015/0094225 A1
(43) Pub. Date: Apr. 2, 2015

(54) METHOD OF OBTAINING INFORMATION FOR IDENTIFYING TUMOR CELL UNDERGOING EPITHELIAL-MESENCHYMAL TRANSITION IN SAMPLE, METHOD OF IDENTIFYING TUMOR CELL UNDERGOING EPITHELIAL-MESENCHYMAL TRANSITION IN SAMPLE, METHOD OF DIAGNOSING SUBJECT HAVING TUMOR CELL UNDERGOING EPITHELIAL-MESENCHYMAL TRANSITION AND COMPOSITION OR KIT FOR IDENTIFYING TUMOR CELL UNDERGOING EPITHELIAL-MESENCHYMAL TRANSITION IN SAMPLE

(71) Applicants: Samsung Electronics Co., Ltd., Suwon-si (KR); Ajou University Industry-Academic Cooperation Foundation, Suwon-si (KR)

(72) Inventors: Yeonjeong Kim, Seoul (KR); Kyungyeon Han, Seoul (KR); Yousun Kim, Suwon-si (KR); Donghyun Park, Chuncheon-si (KR); Jungwon Keum, Seoul (KR); Jongmyeon Park, Incheon (KR)

(21) Appl. No.: 14/504,019

(22) Filed: Oct. 1, 2014

(30) Foreign Application Priority Data
Oct. 1, 2013 (KR) 10-2013-0117594

(51) Int. Cl. C12Q 1/68; G01N 33/574

(52) U.S. Cl. CPC C12Q 1/6886; G01N 33/57496; C12Q 2600/158
USPC 506/9; 435/6.12; 435/7.4

(57) ABSTRACT

Provided are a method of obtaining information for identifying tumor cells undergoing epithelial-mesenchymal transition in a sample, a method of identifying tumor cells undergoing epithelial-mesenchymal transition in a sample, a method of diagnosing a subject having a tumor, and a composition or kit for identifying tumor cells undergoing epithelial-mesenchymal transition in a sample.

### RELATED APPLICATIONS

This application claims the benefit of Korean Patent Application No. 10-2013-0117594, filed on Oct. 1, 2013, the entire disclosure of which is hereby incorporated by reference.

### INCORPORATION BY REFERENCE OF ELECTRONICALLY SUBMITTED MATERIALS

Incorporated by reference in its entirety herein is a computer-readable nucleotide/amino acid sequence listing submitted herewith and identified as follows: One 7,530 bytes ASCII (Text) file named “718577_ST25.TXT” created Oct. 1, 2014.

### BACKGROUND

1. Field

The present disclosure relates to methods of obtaining information for identifying tumor cells undergoing epithelial-mesenchymal transition (EMT-undergoing tumor cells) in a sample, methods of identifying EMT-undergoing tumor cells in a sample, methods of efficiently diagnosing a subject having EMT-undergoing tumor cells, and compositions or kits for identifying EMT-undergoing tumor cells in a sample.

2. Description of the Related Art

Metastatic cancer is the spread of cancer from the region where cancer originated in a body of a living organism to another non-adjacent region. Metastasis influences the choice of therapeutic method to treat cancer and is very important for cancer prognosis. Metastatic cancers are more difficult to treat than primary cancers. Also, metastatic cancers may be tolerant to anti-cancer agents or radiotherapy.

Epithelial-mesenchymal transition (EMT) is a process where epithelial cells lose their cell polarity and cell-cell adhesion and gain migratory and invasive properties to become mesenchymal cells. Epithelial cells are different from mesenchymal cells in phenotypes as well as functions thereof. Epithelial cells are closely connected to each other by tight junctions, gap junctions, and adherens junctions, have an apico-basal polarity and polarization of the actin cytoskeleton, and are bound by a basal lamina at their basal surface. On the other hand, mesenchymal cells lack such polarization, have a spindle shaped morphology, and interact with each other only through focal points. Epithelial cells express high levels of E-cadherein, whereas mesenchymal cells show high levels of N-cadherein, fibronectin, and vimentin.

From a biological point of view, EMT has been categorized into 3 types of developmental (Type I), fibrosis and wound healing (Type II), and cancer (Type III). Initiation of metastasis requires invasion, which is enabled by EMT. Carcinoma cells in primary tumors lose cell-cell adhesion mediated by E-cadherin repression and break through the basement membrane with increased invasive properties and enter the bloodstream through intravasation. Then, when these circulating tumor cells (CTCs) exit the bloodstream and form micrometastases, they undergo mesenchymal-epithelial transition (MET) for clonal outgrowth at these metastatic sites. Thus, EMT and MET form the initiation and completion of the invasion-metastasis cascade. Recently, it has been suggested that cells that undergo EMT gain stem cell-like properties, thus giving rise to cancer stem cells (CSCs).

Thus, there is still a need to detect marker proteins or genes specific to EMT-undergoing cells and to conduct research on the use thereof.

### SUMMARY

Provided are methods of efficiently obtaining information for identifying tumor cells undergoing epithelial-mesenchymal transition (EMT-undergoing tumor cells) in a sample.

Also provided are methods of efficiently identifying EMT-undergoing tumor cells in a sample.

Further provided are methods of efficiently diagnosing a subject having an EMT-undergoing tumor cells.

Yet further provided are compositions for identifying EMT-undergoing tumor cells in a sample.

Additionally provided are kits for identifying EMT-undergoing tumor cells in a sample.

Additional aspects will be set forth in part in the description which follows and, in part, will be apparent from the description, or may be learned by practice of the presented embodiments.

According to an aspect of the present disclosure, a method of obtaining information for identifying tumor cells undergoing epithelial-mesenchymal transition in a sample includes providing a sample comprising a tumor cell obtained from a subject, measuring an expression level of at least one selected from the group consisting of stearoyl-CoA desaturase1 (SCD1), acyl-CoA thioesterase (ACOT1), and protein tyrosine phosphatase-like member B (proline instead of catalytic arginine) (PTPLB) in the sample; and comparing the expression level with that of a control sample.

According to an aspect of the present disclosure, a method of identifying tumor cells undergoing epithelial-mesenchymal transition in a sample includes: providing a sample comprising a tumor cell obtained from a subject; measuring an expression level of at least one selected from the group consisting of stearoyl-CoA desaturase1 (SCD1), acyl-CoA thioesterase (ACOT1), and protein tyrosine phosphatase-like member B (proline instead of catalytic arginine) (PTPLB) in the sample, comparing the expression level with that of a control sample; and identifying tumor cells as undergoing or having undergone epithelial-mesenchymal transition based upon the comparison of the expression level with that of a control sample.

According to an aspect of the present disclosure, a composition for identifying tumor cells undergoing or undergone epithelial-mesenchymal transition in a sample includes a reagent to determine an expression level of at least one selected from the group consisting of stearoyl-CoA desaturase1 (SCD1), acyl-CoA thioesterase (ACOT1), and protein tyrosine phosphatase-like member B (proline instead of catalytic arginine) (PTPLB).

According to an aspect of the present disclosure, a method of diagnosing a subject having a tumor undergoing or undergone epithelial-mesenchymal transition includes: providing a sample comprising a tumor cell obtained from a subject, measuring an expression level of at least one selected from the group consisting of stearoyl-CoA desaturase1 (SCD1), acyl-CoA thioesterase (ACOT1), and protein tyrosine phosphatase-like member B (proline instead of catalytic arginine) (PTPLB) in the sample, comparing the expression level with that of a control sample; and providing a diagnosis to the subject of (not) having tumor cells undergoing EMT or having undergone EMT based upon the comparison of the expression level with that of a control level.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and/or other aspects will become apparent and more readily appreciated from the following description of the embodiments, taken in conjunction with the accompanying drawings in which:

FIG. 1 is a series of micrographs depicting mammospheres formed as a results of mammosphere culture of MCF7 cells;

FIG. 2 is a gel electrophoresis photograph illustrating RT-PCR results of mammosphere-cultured MCF7 cells;

FIG. 3 is a series of fluorescent micrographs illustrating analysis results of mammosphere-cultured MCF7 cells by immunocytochemistry;

FIG. 4 is a series of graphs illustrating analysis results of mammosphere-cultured MCF7 cells by flow cytometry using anti-CD24 and anti-CD44;

FIG. 5 is a photograph illustrating results of a western blotting assay of mammosphere-cultured MCF7 cells;

FIG. 6 are graphs illustrating analysis results of phosphatidyl choline of EMT-induced MCF7 cells using LC-MS;

FIG. 7 is a graph illustrating analysis results of phosphatidyl ethanolamine of EMT-induced MCF7 cells using LC-MS;

FIG. 8 is a graph illustrating analysis results of triglyceride of EMT-induced MCF7 cells using LC-MS;

FIG. 9 is a graph illustrating analysis results of triglyceride of EMT-induced MCF7 cells using LC-MS;

FIG. 10A is a graph illustrating analysis results of ether-linked phosphatidyl ethanolamine of EMT-induced MCF7 cells using LC-MS, wherein the downward arrow indicates a sphere;

FIG. 10B is a graph illustrating analysis results of ether-linked phosphatidyl choline of EMT-induced MCF7 cells using LC-MS;

FIG. 11 is a gel electrophoresis photograph illustrating analysis results of lipogenesis-associated genes in EMT-induced MCF7 cells using RT-PCR;

FIG. 12 are photographs illustrating western blotting results (left) and RT-PCR results (right) of EMT-induced MCF7 cells cultured in a TSA-containing medium;

FIG. 13 is a gel electrophoresis photograph illustrating RT-PCR results of EMT-induced MCF7 cells cultured in a TSA-containing medium;

FIG. 14 is a gel electrophoresis photograph illustrating RT-PCR results of EMT-induced MCF7 cells cultured in a medium including TSA or SB;

FIG. 15 illustrates western blotting results of EMT-induced MCF7 cells cultured in a CoCl₂-containing medium (top) and RT-PCR results (bottom) of EMT-induced MCF7 cells cultured in a CoCl₂-containing medium;

FIG. 16 is a gel electrophoresis photograph illustrating RT-PCR results of EMT-induced MCF7 cells cultured in a CoCl₂-containing medium;

FIG. 17 illustrates western blotting results of EMT-induced lung cancer cell lines NCI-1650 cells cultured in a TSA-containing medium (A); and RT-PCR results of EMT-induced lung cancer cell lines NCI-1650 cells cultured in a TSA-containing medium (B); and

FIG. 18 illustrates western blotting results of EMT-induced prostate cancer cell line DU145 cells cultured in a medium including TSA, SB, or CoCl₂ (A); and RT-PCR results of EMT-induced prostate cancer cell line DU145 cells cultured in a medium including TSA, SB, or CoCl₂ (B).

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being in any way limited to the descriptions set forth herein. Accordingly, the embodiments are merely described below, by referring to the figures, to aid in elucidating aspects of the present description. As used herein, the term “and/or” includes any and all combinations of one or more of the associated listed items. Expressions such as “at least one of,” when preceding a list of elements, modify the entire list of elements and do not modify the individual elements of the list.

According to an embodiment of the present disclosure, provided is a method of obtaining information for identifying tumor cells undergoing or undergone epithelial-mesenchymal transition (hereinafter, referred to as EMT-undergoing tumor cells) in a sample, the method including providing a sample comprising a tumor cell obtained from a subject, measuring in the sample an expression level of at least one selected from the group consisting of stearoyl-CoA desaturase1 (SCD1), acyl-CoA thioesterase (ACOT1), and protein tyrosine phosphatase-like member B (proline instead of catalytic arginine) (PTPLB), and comparing the expression level with that of a control sample.

The method of obtaining information for identifying EMT-undergoing tumor cells in a sample includes providing a sample comprising a tumor cell obtained from a subject. The subject may be a living organism or an isolate from a living organism or tumor cell containing medium. The living organism may be a mammal. The mammal may be a human, rat, cow, horse, sheep, or pig. The isolate may be a tumor cell separated from the living organism such as biopsy, and tissue therefrom. The tumor cell containing medium may include in vitro tumor cell suspension such as tumor cell culture. The sample may include cells. The sample may be a sample derived from a living organism. For example, the sample may include a tumor tissue, blood, bone marrow, lymph, saliva, tears, urine, mucus, amniotic fluid, or any combination thereof. The sample may be isolated from a subject.

The cells in the sample may be cancer cells. The cancer cells may be circulating tumor cells (CTCs), breast cancer cells, prostate cancer cells, lung cancer cells, colorectal cancer cells, gastric cancer cells, ovarian cancer cells, endometrial cancer cells, liver cancer cells, esophageal cancer cells, pancreatic cancer cells, or thyroid cancer cells,

The control sample may be a sample including normal cells, tumor cells that have not undergone epithelial-mesenchymal transition, or any combination thereof (hereinafter referred to “negative control sample”). The control sample may be a sample including tumor cells that have undergone EMT (hereinafter referred to “positive control sample”). The control sample may be isolated from a subject. The subject may be a mammal. The mammal may be a human, rat, cow, horse, sheep, or pig. The control sample may include in vitro cultured tumor cells.

The method of obtaining information for identifying EMT-undergoing tumor cells in a sample includes measuring an expression level of at least one selected from the group consisting of stearoyl-CoA desaturase1 (SCD1), acyl-CoA thioesterase (ACOT1), and protein tyrosine phosphatase-like member B (proline instead of catalytic arginine) (PTPLB) in the sample. The terms “expression level” includes the expressed amount of a gene in terms of protein, nucleic acid such as mRNA, or a combination thereof. The measuring the expression level may be performed by using known methods. For example, the measuring may be measuring of an expression level at the protein level by quantifying at least one of SCD1, ACOT1, and PTPLB. The quantifying may be performed by immunoassays. The immunoassay may include radioimmunoassay, radioimmunoprecipitation, immunoprecipitation, enzyme-linked immunosorbent assay (ELISA), capture-ELISA, inhibition or competition assay, sandwich assay, flow cytometry, immunofluorescence, and immunoaffinity purification. An antibody specifically binding to the protein may be used in the immunoassay. In addition, the protein may be directly isolated from cells and quantified. The isolation may be performed using centrifugation, filtration, chromatography, precipitation, electrophoresis, dialysis, crystallization, or any combination thereof. The chromatography may include affinity chromatography, size exclusion chromatography, ion-exchange chromatography, or any combination thereof.

The measuring the expression level may be measuring an amount of a nucleic acid such as mRNA encoding the protein. The mRNA may be quantified by nucleic acid amplification. The amplification of nucleic acid may include techniques that require multiple thermal cycling during the amplification or techniques that are performed at a single temperature. Cycling techniques may include techniques that require thermal cycling. Techniques requiring thermal cycling include polymerase chain reaction (PCR). PCR is well known in the art. The PCR includes denaturing a double-stranded DNA into single stranded DNAs by thermal denaturation, annealing primers to the single stranded DNAs; and synthesizing complementary strands from the primer. Isothermal amplification is an amplification performed without a thermal cycling for example, at a single temperature or where the major aspect of the amplification process is performed at a single temperature. Isothermal techniques rely on a strand displacing polymerase to separate two strands of a double strand and re-copy templates. Isothermal techniques may be classified into methods that rely on the replacement of a primer to initiate a reiterative template copying and those that rely on continued re-use or new synthesis of a single primer molecule. The methods that rely on the replacement of the primer include a helicase dependent amplification (HDA), an exonuclease dependent amplification, a recombinase polymerase amplification (RPA), and a loop mediated amplification (LAMP). The methods that rely on continued re-use or new synthesis of a single primer molecule include a strand displacement amplification (SDA) or a nucleic acid based amplification (NASBA and TMA). The amplification may be connected to reverse-transcribing reaction such as RT-PCR. The measuring may include directly isolating the nucleic acid such as mRNA and quantifying the amount by known method such as spectrophotometry including absorbance at a wavelengths 260 nm and 280 nm. The measuring may include hybridizing the nucleic acid with a probe and detecting the hybridization products.

Stearoyl-CoA desaturase 1 (SCD1) is a protein encoded by SCD gene in humans. SCD1 is a key enzyme in fatty acid metabolism. This enzyme forms a double bond in stearoyl-CoA. As a result, oleic acid, which is mono-unsaturated fatty acid, may be produced from stearic acid, which is saturated fatty acid. SCD may be an enzyme belonging to enzyme code (EC) 1.14.19.1 and may be an iron-containing enzyme catalyzing a rate-limiting operation in the synthesis of unsaturated fatty acids. SCD1 may have an amino acid sequence of RefSeq NP_{—}005054 or NP_{—}033153. SCD1 may be encoded by a nucleotide sequence of RefSeq NM_{—}005063 or NM_{—}009127.

Acyl-CoA thioesterase 1 (ACOT1) is an enzyme that is found in cytoplasm and hydrolyzes long chain acyl-CoA of C12-C20-CoA in chain-length into free fatty acid and CoA. ACOT1 may be an enzyme belonging to EC 3.1.2.2. ACOT1 may have an amino acid sequence of RefSeq NP_{—}001032238.1. ACOT1 may be encoded by a nucleotide sequence of RefSeq NM_{—}001037161.1.

Protein tyrosine phosphatase-like member B (PTPLB) is an intracellular protein of endoplasmic reticulum membrane and is also known as BAP31-interacting protein or long-chain (3R)-3-hydroxyacyl-[acyl-carrier-protein] dehydratase 2. PTPLB may have an amino acid sequence of RefSeq NP_{—}940684.1. PTPLB may be encoded by a nucleotide sequence of RefSeq NM_{—}198402.3.

The method of obtaining information for identifying EMT-undergoing tumor cells in a sample includes comparing the expression level with that of a control sample. The expression level in terms of protein may be compared with the expression level in terms of protein of a control sample.

The method may further include measuring an expression level of at least one selected from the group consisting of acetyl-CoA carboxylase alpha (ACACA), fatty acid synthase (FASN), peroxisomal trans-2-enoyl-CoA reductase (PECR), elongation of very long chain fatty acids (FEN1/Elo2, SUR4/Elo3, yeast)-like 2 (ELOVL2), elongation of very long chain fatty acids (FEN1/Elo2, SUR4/Elo3, yeast)-like 3 (ELOVL3), and peroxisome proliferator activator receptor-gamma (PPARγ) in the sample; and comparing the expression level with that of a control sample. The terms “expression level” includes the expressed amount of a gene in terms of protein, nucleic acid such as mRNA, or a combination thereof. The measuring the expression level may be performed by using known methods. For example, the measuring may be measuring of an expression level at the protein level by quantifying at least one of ACACA, FASN, PECR, ELOVL2, ELOVL3, and PPARγ. The quantifying may be performed by immunoassays. The immunoassay may include radioimmunoassay, radioimmunoprecipitation, immunoprecipitation, enzyme-linked immunosorbent assay (ELISA), capture-ELISA, inhibition or competition assay, sandwich assay, flow cytometry, immunofluorescence, and immunoaffinity purification. An antibody specifically binding to the protein may be used in the immunoassay. In addition, the protein may be directly isolated from cells and quantified. The isolation may be performed using centrifugation, filtration, chromatography, precipitation, electrophoresis, dialysis, crystallization, or any combination thereof. The chromatography may include affinity chromatography, size exclusion chromatography, ion-exchange chromatography, or any combination thereof.

ACACA is encoded by ACACA gene in humans. Acetyl-CoA carboxylase (ACC) is a complex multi-functional enzyme system. ACC may be a biotin-containing enzyme catalyzing carboxylation of acetyl-CoA that is a rate-limiting operation of synthesis of fatty acid to produce malonyl-CoA. ACACA may have an amino acid sequence of RefSeq NP_{—}942131 (human) or NP_{—}579938 (rat). ACACA may be encoded by a nucleotide sequence of RefSeq NM_{—}000664 (human) or NM_{—}133360 (rat). ACACA may belong to EC 6.3.4.14 and/or 6.4.1.2.

FASN may be an enzyme encoded by FASN gene in humans. FASN may be a multienzyme protein that catalyzes synthesis of fatty acid. FASN is not a single enzyme but a whole enzymatic system composed of two identical 272 kDa multi-functional polypeptides by which a substrate is delivered from one functional domain to another functional domain. The main function of FASN is to catalyze the synthesis of palmitate, which is a long-chain saturated fatty acid, from acetyl-CoA and malonyl-CoA in the presence of NADPH. FASN may have an amino acid sequence of RefSeq NP_{—}004095.4 (human) or NP_{—}032014.3 (rat). FASN may be encoded by a nucleotide sequence of RefSeq NM_{—}004104.4 (human) or NM_{—}007988.3 (rat).

PECR participates in chain elongation of fatty acids and may be located in peroxisome. PECR may have an amino acid sequence of RefSeq NP_{—}060911.2. PECR may be encoded by a nucleotide sequence of RefSeq NM_{—}018441.4.

ELOVL2 may participate in tissue-specific synthesis of very long chain fatty acids and sphingolipids and may be located in endoplasmic reticulum membrane. ELOVL2 may be an enzyme belonging to EC 2.3.1.199. ELOVL2 may have an amino acid sequence of RefSeq NP_{—}060240.3. ELOVL2 may be encoded by a nucleotide sequence of RefSeq NM_{—}017770.3.

ELOVL3 may participate in tissue-specific synthesis of very long chain fatty acids and sphingolipids and may be located in endoplasmic reticulum membrane. ELOVL2 may be an enzyme belonging to EC 2.3.1.199. ELOVL3 may have an amino acid sequence of RefSeq NP_{—}689523.1. ELOVL2 may be encoded by a nucleotide sequence of RefSeq NM_{—}152310.1.

PPARγ may be a type II nuclear receptor encoded by PPARG gene in humans. PPARγ is expressed as two isomers in the humans and rats. PPARγ1 is found in almost all tissues except muscle, and PPARγ2 is found in adipose tissue and the intestine. PPARγ may have an amino acid sequence of RefSeq NP_{—}005028 (human) or NP_{—}001120802 (rat). PPARγ may be encoded by a nucleotide sequence of RefSeq NM_{—}005037 (human) or NM_{—}001127330 (rat).

The method may further include isolating tumor cells from the sample before the determining of the expression level of at least one of the proteins. For example, the isolation of the tumor cells may be performed by using a substance specifically binding to a tumor-specific cell surface marker, by specifically isolating tumor cells based on sizes thereof, or by using flow cytometry such as FACS. For example, a tumor cell may be separated from the sample, by contacting a sample including tumor cells with a solid support to which a substance specifically binding to a tumor-specific cell surface marker, such as an antibody or an antigen-binding fragment thereof is immobilized, to form a tumor cell-support complex, isolating the tumor cell-support complex from the reaction mixture, and selectively isolating the tumor cells from the tumor cell-support complexes. The solid support may have various shapes such as a spherical shape, a bead-like shape, or a plate-like shape. The solid support may also be magnetic particles. The particles may be nano or micro particles. The isolating of the tumor cells may be an isolation of CTCs. The isolation of CTCs may be performed by using a CTC-specific surface marker, such as epithelial cell adhesion molecule (EpCAM), discoidin domain receptor (DDR)1, Her2, PSA, or any combination thereof. As a result, by identifying the expression level of the protein in the isolated CTCs, it may be determined whether the CTCs are EMT-undergoing CTCs or EMT-undergoing breast cancer cells.

The method may further include measuring a relative expression level of at least one mono-unsaturated phospholipid and at least one poly unsaturated phospholipid in cells of the sample with respect to a sample including normal cells. In this regard, an increase in the relative expression level of the at least one mono-unsaturated phospholipid and a reduction in the relative expression level of the at least one poly unsaturated phospholipid may indicate a cancer having an aggressive lipogenic phenotype. The measuring may include measuring the amount of at least one mono-unsaturated phospholipid and at least one poly unsaturated phospholipid in the sample and in a sample including normal cells and comparing the amount in the sample with that in the control sample. The measuring may be performed using high performace liquid chromatography (HPLC).

EMT-undergoing tumor cells may be metastatic cancer cells or cancer stem cells (CSCs). The method of obtaining information for identifying EMT-undergoing tumor cells in a sample may be in vitro method.

According to another embodiment of the present disclosure, there is provided a method of identifying EMT-undergoing tumor cells in a sample, the method including: providing a sample comprising a tumor cell obtained from a subject, measuring an expression level of at least one selected from the group consisting of stearoyl-CoA desaturase1 (SCD1), acyl-CoA thioesterase (ACOT1), and protein tyrosine phosphatase-like member B (PTPLB, proline instead of catalytic arginine) in the sample, comparing the expression level with that of a control sample; and identifying tumor cells as undergoing or having undergone EMT based upon the comparison of the expression level with that of the control.

Identifying whether tumor cells are undergoing or have undergone EMT may include determining that the cells are EMT-undergoing tumor cells when, in comparison with expression levels of the control sample, at least one of a higher expression level of SCD1, a lower expression level of ACOT1, and a lower expression level of PTPLB is observed in the tumor cells. Identifying whether tumor cells are undergoing or have undergone EMT may include determining that the cells are not EMT-undergoing tumor cells when at least one of a lower expression level of SCD1, a higher expression level of ACOT1, and a higher expression level of PTPLB than those of the control sample is observed.

The method includes providing a sample comprising a tumor cell obtained from a subject. The subject may be a living organism or an isolate from a living organism or tumor cell containing medium. The living organism may be a mammal. The mammal may be a human, rat, cow, horse, sheep, or pig. The isolate may be a tumor cell containing detached from the living organism such as biopsy, and tissue. The tumor cell containing medium may include in vitro tumor cell suspension such as tumor cell culture. The sample may include cells. The sample may be a sample derived from a living organism. For example, the sample may include a tumor tissue, blood, bone marrow, lymph, saliva, tears, urine, mucus, amniotic fluid, or any combination thereof. The sample may be isolated from a subject. The cells in the sample may be cancer cells. The cancer cells may be breast cancer cells, prostate cancer cells, lung cancer cells, colorectal cancer cells, gastric cancer cells, ovarian cancer cells, endometrial cancer cells, liver cancer cells, esophageal cancer cells, pancreatic cancer cells, or thyroid cancer cells,

The control sample may be a sample including normal cells, tumor cells that have not undergone epithelial-mesenchymal transition, or any combination thereof (hereinafter referred to “negative control sample”). The control sample may be a sample including tumor cells that does not undergone EMT (hereinafter referred to “positive control sample”). The control sample may be isolated from a subject. The subject may be a mammal. The mammal may be a human, rat, cow, horse, sheep, or pig. The control sample may include in vitro cultured tumor cells.

The method includes measuring an expression level of at least one selected from the group consisting of stearoyl-CoA desaturase1 (SCD1), acyl-CoA thioesterase (ACOT1), and protein tyrosine phosphatase-like member B (proline instead of catalytic arginine) (PTPLB) in the sample. The terms “expression level” includes the expressed amount of a gene in terms of protein, nucleic acid such as mRNA, or a combination thereof. The measuring the expression level may be performed by use of known methods. For example, the measuring may be measuring an expression level at the protein level by quantifying at least one of SCD1, ACOT1, and PTPLB. The quantifying may be performed by immunoassays. The immunoassay may include radioimmunoassay, radioimmunoprecipitation, immunoprecipitation, enzyme-linked immunosorbent assay (ELISA), capture-ELISA, inhibition or competition assay, sandwich assay, flow cytometry, immunofluorescence, and immunoaffinity purification. An antibody specifically to the protein may be used in the immunoassay. In addition, the protein may be directly isolated from cells and quantified. The isolation may be performed using centrifugation, filtration, chromatography, precipitation, electrophoresis, dialysis, crystallization, or any combination thereof. The chromatography may include affinity chromatography, size exclusion chromatography, ion-exchange chromatography, or any combination thereof.

The expression level may also be determined by transporting the protein or a substance specifically binding to a nucleic acid encoding the protein into cytoplasm or nuclei through cell membranes and/or nuclear membrane, staining the protein or substance in the cells, and visualizing the stained cells. The transportation into the cytoplasm or nuclei may be performed by permeabilization of cell membranes or nuclear membranes. The specifically binding substance may be an antibody specifically binding to the protein, an antigen-binding fragment thereof, or a nucleic acid specifically binding to a transcript of a polynucleotide encoding the protein, for example a mRNA or a complement thereof. The specifically binding substance may be labeled with a detectable substance such a fluorescent substance.

The method includes comparing the expression level with that of a control sample. The expression level in terms of protein may be compared with the expression level in terms of protein of a control sample.

The method includes determining that the cells are EMT-undergoing tumor cells when at least one selected from the group consisting of a higher expression level of SCD1, a lower expression level of ACOT1, and a lower expression level of PTPLB than those of the control sample is observed. It was confirmed that the expression of SCD1 increases and the expressions of ACOT1 and PTPLB are reduced, when tumor cells undergo epithelial-mesenchymal transition, as described above.

The method may further include measuring an expression level of at least one selected from the group consisting of acetyl-CoA carboxylase alpha (ACACA), fatty acid synthase (FASN), peroxisomal trans-2-enoyl-CoA reductase (PECR), elongation of very long chain fatty acids (FEN1/Elo2, SUR4/Elo3, yeast)-like 2 (ELOVL2), elongation of very long chain fatty acids (FEN1/Elo2, SUR4/Elo3, yeast)-like 3 (ELOVL3), and peroxisome proliferator activator receptor-gamma (PPARγ) in the sample, and comparing the expression level with that of the control sample. The terms “expression level” includes the expressed amount of a gene in terms of protein, nucleic acid such as mRNA, or a combination thereof. The measuring the expression level may be performed by using known methods. For example, the measuring may be measruing of an expression level at the protein level by quantifying at least one of ACACA, FASN, PECR, ELOVL2, ELOVL3, and PPARγ. The quantifying may be performed by immunoassays. The immunoassay may include radioimmunoassay, radioimmunoprecipitation, immunoprecipitation, enzyme-linked immunosorbent assay (ELISA), capture-ELISA, inhibition or competition assay, sandwich assay, flow cytometry, immunofluorescence, and immunoaffinity purification. An antibody specifically binding to the protein may be used in the immunoassay. In addition, the protein may be directly isolated from cells and quantified. The isolation may be performed using centrifugation, filtration, chromatography, precipitation, electrophoresis, dialysis, crystallization, or any combination thereof. The chromatography may include affinity chromatography, size exclusion chromatography, ion-exchange chromatography, or any combination thereof.

The determining of the expression level of the protein is as described above.

The method includes determining that the cells are EMT-undergoing breast cancer cells when at least one selected from the group consisting of a higher expression level of ACACA, a higher expression level of FASN, a lower expression level of PECR, a lower expression level of ELOVR2, a lower expression level of ELOVR3, and a lower expression level of PPARγ than those of the control sample is observed.

The method may further include isolating tumor cells from the sample before the determining of the expression level of at least one of the proteins. For example, the isolation of the tumor cells may be performed by using a substance specifically binding to a tumor-specific cell surface marker, by specifically isolating tumor cells based on sizes thereof, or by using flow cytometry such as FACS. For example, a tumor cell may be separated from the sample, by contacting a sample including tumor cells with a solid support to which a substance specifically binding to a tumor-specific cell surface marker, such as an antibody or an antigen-binding fragment thereof, is immobilized, to form a tumor cell-support complexes, isolating the tumor cell-support complexes from the reaction mixture, and selectively isolating the tumor cells from the tumor cell-support complexes. The solid support may have various shapes such as a spherical shape, a bead-like shape, or a plate-like shape. The solid support may also be magnetic particles. The particles may be nano or micro particles. The isolating of the tumor cells may be an isolation of CTCs. The isolation of CTCs may be performed by using a CTC-specific surface marker, such as epithelial cell adhesion molecule (EpCAM), discoidin domain receptor (DDR)1, Her2, PSA, or any combination thereof. As a result, by identifying the expression of the protein in the isolated CTCs, it may be determined whether the CTCs are EMT-undergoing CTCs or EMT-undergoing breast cancer cells.

The method may further include measuring a relative expression level of at least one mono-unsaturated phospholipid and at least one poly unsaturated phospholipid in cells of the sample with respect to a sample of normal cells. In this regard, an increase in the relative expression level of the at least one mono-unsaturated phospholipid and a reduction in the relative expression level of the at least one poly-unsaturated phospholipid may indicate a cancer having an aggressive lipogenic phenotype. The measuring may include measuring the amount of at least one mono-unsaturated phospholipid and at least one poly unsaturated phospholipid in the sample and in a sample including normal cells and comparing the amount in the sample with that in the control sample. The measuring may be performed using high performance liquid chromatography (HPLC).

EMT-undergoing tumor cells may be metastatic cancer cells or cancer stem cells (CSCs). The method of identifying EMT-undergoing tumor cells in a sample may be in vitro method.

According to another embodiment of the present disclosure, provided is a method of diagnosing a subject having a tumor undergoing or undergone EMT, the method including: providing a sample comprising a tumor cell obtained from a subject, measuring an expression level of at least one selected from the group consisting of stearoyl-CoA desaturase1 (SCD1), acyl-CoA thioesterase (ACOT1), and protein tyrosine phosphatase-like member B (PTPLB, proline instead of catalytic arginine) in the sample, comparing the expression level with that of a control sample; and diagnosing the subject as having tumor cells undergoing EMT or which have undergone EMT based upon the comparison of the expression level with that of a control sample. The diagnosing of the subject as having tumor cells undergoing EMT or which have undergone EMT may include determining that the cells are EMT-undergoing tumor cells when at least one selected from the group consisting of a higher expression level of SCD1, a lower expression level of ACOT1, and a lower expression level of PTPLB than those of the control sample is observed, wherein the control sample is normal cells, or tumor cells that do not undergo EMT. The steps of “providing a sample comprising a tumor cell obtained from a subject”, “measuring” and “comparing” may be as described above.

The method may further include: measuring an expression level of at least one selected from the group consisting of acetyl-CoA carboxylase alpha (ACACA), fatty acid synthase (FASN), peroxisomal trans-2-enoyl-CoA reductase (PECR), elongation of very long chain fatty acids (FEN1/Elo2, SUR4/Elo3, yeast)-like 2 (ELOVL2), elongation of very long chain fatty acids (FEN1/Elo2, SUR4/Elo3, yeast)-like 3 (ELOVL3), and peroxisome proliferator activator receptor-gamma (PPARγ) in the sample, comparing the expression level with that of the control sample; and the providing tumor cells undergoing or undergone EMT diagnosis includes determining that the cells are EMT-undergoing cancer cells when at least one selected from the group consisting of a higher expression level of ACACA, a higher expression level of FASN, a lower expression level of PECR, a lower expression level of ELOVR2, a lower expression level of ELOVR2, and a lower expression level of PPARγ in the cells than those of the control sample is observed. The diagnosing a subject as having tumor cells undergoing EMT or which have undergone EMT may include determining that the subject does not have tumor cells undergoing or undergone epithelial-mesenchymal transition when at least one selected from the group consisting of a lower expression level of ACACA, a lower expression level of FASN, a higher expression level of PECR, a higher expression level of ELOVR2, a higher expression level of ELOVR3, and a higher expression level of PPARγ than those of the control sample is observed, wherein the control sample is normal cells, or tumor cells that do not undergo EMT. The steps of “measuring” and “comparing” is as described above.

The subject may be a mammal. The mammal may be a human, rat, cow, horse, sheep, or pig.

The method of diagnosing a subject having a tumor cancer undergoing or undergone EMT may be an in vitro method. The method of diagnosing a subject having a tumor undergoing or undergone EMT may further include administering one or more drugs for treating a cancer to a subject diagnosed as having a cancer or higher possibility of having a cancer according to the method of diagnosing cancer to treat a cancer. The cancer may be a cancer including the tumor cells undergoing or undergone EMT. The drugs may be anti-cancer drug including for example, anti-cancer drug specific for a cancer including the tumor cells undergoing or undergone EMT, or combination with other cancer drugs.

According to another embodiment of the present disclosure, provided is a composition for identifying EMT-undergoing tumor cells in a sample including a reagent to determine an expression level of at least one selected from the group consisting of SCD1, ACOT1, and PTPLB.

The reagent may include a substance binding to at least one of the SCD1, ACOT1, and PTPLB or may be a reagent used to amplify nucleotides encoding at least one of the SCD1, ACOT1, and PTPLB.

The binding substance may be an antibody, a functional fragment thereof, a ligand, a receptor, a substrate, an agonist, an antagonist, an inhibitor, or any combination thereof. The nucleotide may be mRNA. The reagent may include a nucleotide segment derived from a nucleotide sequence encoding at least one of the SCD1, ACOT1, and PTPLB or a nucleotide sequence complementary thereto. The nucleotide segment may be at least 10 bp, 15 bp, 20 bp, 30 bp, 40 bp, or 50 by in lengths. For example, the nucleotide segment may be 10-100 bp, 15-100 bp, 20-100 bp, 30-100 bp, 40-100 bp, 50-100 bp, 10-80 bp, 15-80 bp, 20-80 bp, 30-80 bp, 40-80 bp, 50-80 bp, 10-60 bp, 15-60 bp, 20-60 bp, 30-60 bp, 40-60 bp, or 50-60 bp in lengths. The reagent may include a primer or a probe.

The composition may further include a reagent to determine an expression level of at least one of the ACACA, FASN, PECR, ELOVL2, ELOVL3, and PPARγ.

The reagent may be a reagent including a substance binding to at least one of the ACACA, FASN, PECR, ELOVL2, ELOVL3, and PPARγ or may be a reagent used to amplify nucleotides encoding at least one of the ACACA, FASN, PECR, ELOVL2, ELOVL3, and PPARγ.

The binding substance may be an antibody, a functional fragment thereof, a ligand, a receptor, a substrate, an agonist, an antagonist, an inhibitor, or any combination thereof. The nucleotide may be mRNA. The reagent may include a nucleotide segment derived from a nucleotide sequence encoding at least one of the ACACA, FASN, PECR, ELOVL2, ELOVL3, and PPARγ or a nucleotide sequence complementary thereto. The nucleotide segment may be at least 10 bp, 15 bp, 20 bp, 30 bp, 40 bp, or 50 by in lengths. For example, the nucleotide segment may be 10-100 bp, 15-100 bp, 20-100 bp, 30-100 bp, 40-100 bp, 50-100 bp, 10-80 bp, 15-80 bp, 20-80 bp, 30-80 bp, 40-80 bp, 50-80 bp, 10-60 bp, 15-60 bp, 20-60 bp, 30-60 bp, 40-60 bp, or 50-60 bp in lengths. The reagent may include a primer or a probe.

According to another embodiment of the present disclosure, provided is a kit for identifying EMT-undergoing tumor cells in a sample including a reagent to determine an expression level of at least one selected from the group consisting of SCD1, ACOT1, and PTPLB.

The reagent may include a substance binding to at least one of the SCD1, ACOT1, and PTPLB or may be a reagent used to amplify nucleotides encoding at least one of the SCD1, ACOT1, and PTPLB.

The binding substance may be an antibody, a functional fragment thereof, a ligand, a receptor, a substrate, an agonist, an antagonist, an inhibitor, or any combination thereof. The nucleotide may be mRNA. The reagent may include a nucleotide segment derived from a nucleotide sequence encoding at least one of the SCD1, ACOT1, and PTPLB or a nucleotide sequence complementary thereto. The nucleotide segment may be at least 10 bp, 15 bp, 20 bp, 30 bp, 40 bp, or 50 bp in lengths. For example, the nucleotide segment may be 10-100 bp, 15-100 bp, 20-100 bp, 30-100 bp, 40-100 bp, 50-100 bp, 10-80 bp, 15-80 bp, 20-80 bp, 30-80 bp, 40-80 bp, 50-80 bp, 10-60 bp, 15-60 bp, 20-60 bp, 30-60 bp, 40-60 bp, or 50-60 bp in lengths. The reagent may include a primer or a probe.

The composition may further include a reagent to determine an expression level of at least one of the ACACA, FASN, PECR, ELOVL2, ELOVL3, and PPARγ.

The reagent may be a reagent including a substance binding to at least one of the ACACA, FASN, PECR, ELOVL2, ELOVL3, and PPARγ or may be a reagent used to amplify nucleotides encoding at least one of the ACACA, FASN, PECR, ELOVL2, ELOVL3, and PPARγ.

The binding substance may be an antibody, a functional fragment thereof, a ligand, a receptor, a substrate, an agonist, an antagonist, an inhibitor, or any combination thereof. The nucleotide may be mRNA. The reagent may include a nucleotide segment derived from a nucleotide sequence encoding at least one of the ACACA, FASN, PECR, ELOVL2, ELOVL3, and PPARγ or a nucleotide sequence complementary thereto. The nucleotide segment may be at least 10 bp, 15 bp, 20 bp, 30 bp, 40 bp, or 50 by in lengths. For example, the nucleotide segment may be 10-100 bp, 15-100 bp, 20-100 bp, 30-100 bp, 40-100 bp, 50-100 bp, 10-80 bp, 15-80 bp, 20-80 bp, 30-80 bp, 40-80 bp, 50-80 bp, 10-60 bp, 15-60 bp, 20-60 bp, 30-60 bp, 40-60 bp, or 50-60 bp in lengths. The reagent may include a primer or a probe.

These examples are for illustrative purposes only and are not intended in any way to limit the scope of the invention.

### Example 1

### EMT Induction of Tumor Cells and Lipid Distribution and Gene Expression Analysis in EMT-Induced Tumor Cells

EMT was induced in various non-metastatic tumor cells, and lipid distribution and gene expression associated thereto in the EMT-induced tumor cells were identified.

1. EMT Induction in Breast Cancer, Prostate Cancer, and Lung Cancer Cell Lines

A Breast cancer cell line MCF-7 (ATCC® HTB-22TM), lung cancer cell lines HCC827 (ATCC Cat. No. CRL-2868) and H1650 (ATCC Cat. No. CRL-5883), and prostate cancer cell lines DU145 and PC3 were purchased from American Type Culture Collection (ATCC). MCF-7 is a non-metastatic primary tumor having an estrogen receptor. HCC827 that is adenocarcinoma is a primary tumor derived from lung epithelial cells.

In order to induce EMT in the cell lines, mammosphere cultures were performed instead of adherent cultures in Dulbecco's Modified Eagle Media (DMEM) supplemented with 10% fetal bovine serum (FBS).

Before performing the mammosphere cultures, each of the cell lines was adherent-cultured in the DMEM supplemented with 10% FBS in a 100 mm tissue culture dish as a monolayer (hereinafter, referred to as “adherent culture”). The cultures were performed in a humidified incubator with 5% CO₂ at 37° C. When the cells were grown up to 80-90% confluent monolayer, the cells were separated therefrom using a cell scraper and washed. Then, the cells were resuspended in DMEM-F12 that is a medium for mammosphere culture supplemented with 1xB27 as a supplementary material for cell growth, 20 ng fibroblast growth factor (FGF)/ml, 20 ng epidermal growth factor (EGF)/ml, and 5 μg insulin/ml.

Then, the resuspended cells were seeded on each well of a 100 mm ultra-low attachment surface dish (Corning, Catalog 3262) to 2×10⁵ cells in 10 ml, and then cultured for 24 hours to 3 weeks. The cultures were performed in a humidified incubator with 5% CO₂ at 37° C. The culture medium was removed once a week by centrifugation, the cells were washed with PBS, and 1 ml of accutase (Invitrogen, Cat. A111050) was added to the obtained cell pellets. Then, the cells were incubated at 37° C. for 5 to 10 minutes to dissociate a lump of cells into single cells, and serially subcultured for 7 to 10 days.

The mammosphere culture was performed for one month, and then the cells from a mammosphere were analysed using RT-PCR and immunocytochemistry to identify markers indicating EMT-undergoing cells.

FIG. 1 illustrates mammospheres formed as results of mammosphere culture of MCF7 cells. As illustrated in FIG. 1, the size of the mammosphere gradually increases over time.

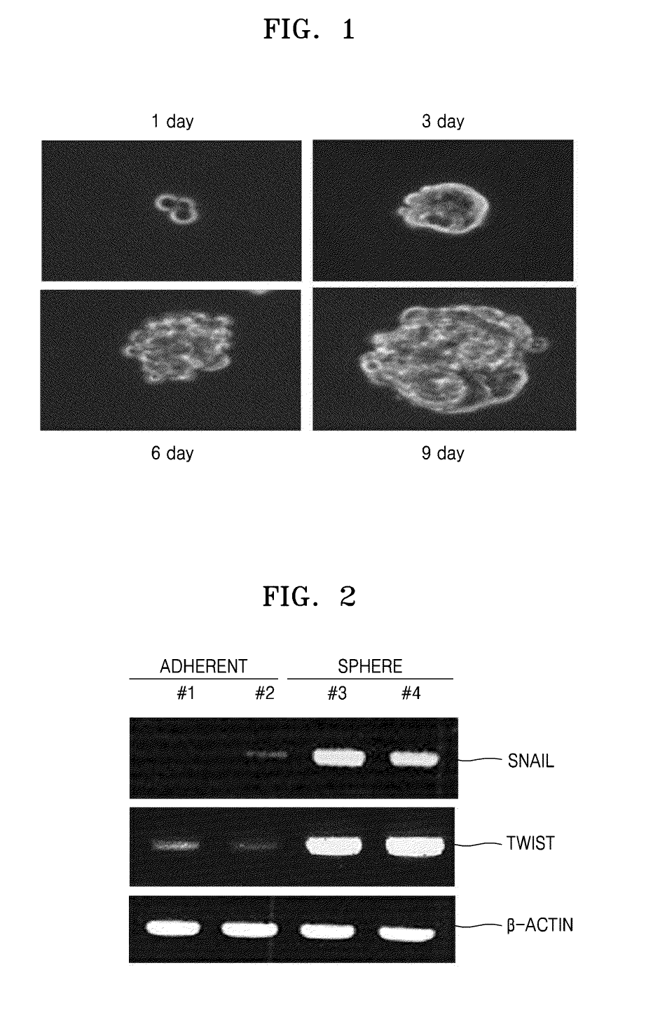

FIG. 2 illustrates RT-PCR results of mammosphere-cultured MCF7 cells. As illustrated in FIG. 2, expressions of Snail and Twist, which are EMT markers, were significantly increased by mammosphere culture in Lanes 3 and 4 in comparison with control groups in Lanes 1 and 2. The control group includes adherent-cultured cells.

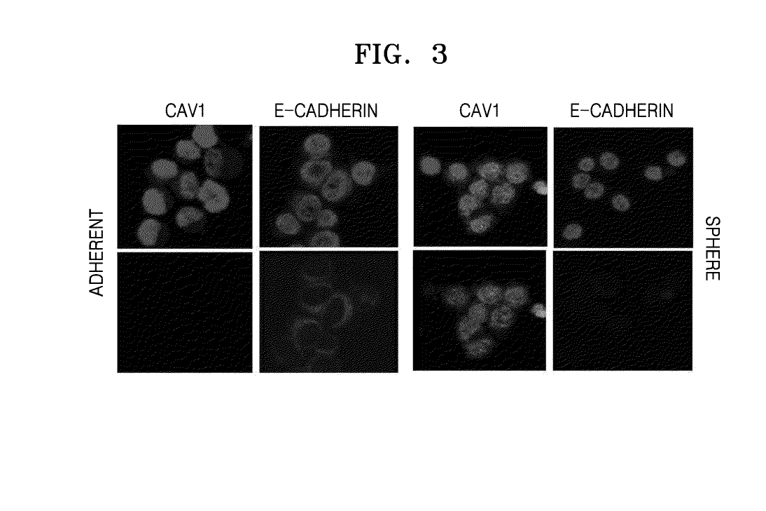

FIG. 3 illustrates analysis results of mammosphere-cultured MCF7 cells by immunocytochemistry. In FIG. 3, a top layer shows results at the beginning and a bottom layer shows results 10 days after culturing. As illustrated in FIG. 3, differently from the adherent-cultured cells of the control group, an expression of caveolin-1 (CAV1) was maintained and an expression of E-cadherin was significantly reduced in the mammosphere-cultured cells. Thus, it was confirmed that EMT was in progress.

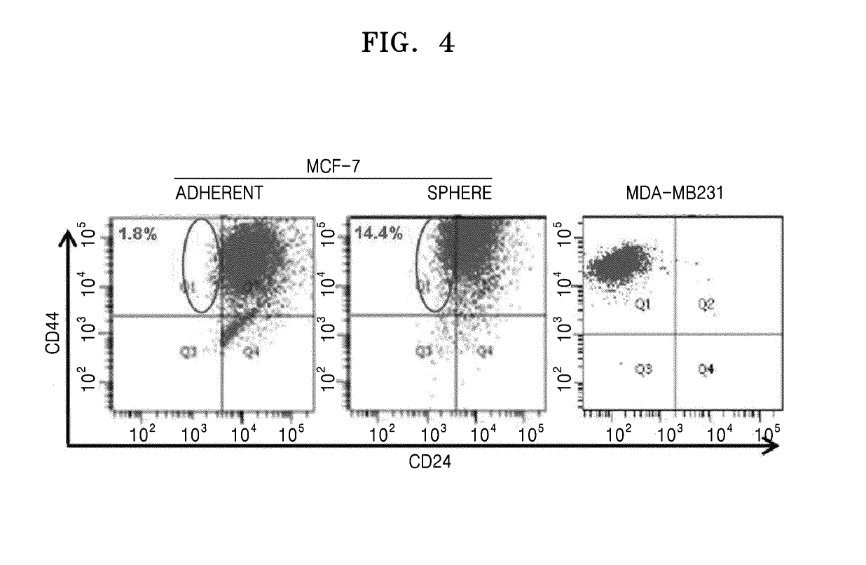

FIG. 4 illustrates analysis results of mammosphere-cultured MCF7 cells by flow cytometry using anti-CD24 and anti-CD44. In FIG. 4, adherent (Control Group 1) indicates adherent-cultured MCF7 cells, and MDA-MB231 (Control Group 2) indicates MDA-MB-231 breast cancer cell lines which were adherent-cultured under the same conditions as those used for adherent-culture of MCF7 cells. MDA-MB-231 breast cancer cell lines have epithelial-like morphology and appear phenotypically as spindle shaped cells. MDA-MB-231 breast cancer cell lines are metastatic and invasive cancer cells. CD24 and CD44 are markers for cancer stem cells (CSCs). As illustrated in FIG. 4, as a result of mammosphere culture, Q1 cells were increased in MCF7 cells compared to those of adherent-cultured cells. Thus, it was confirmed that MCF7 cells transformed into metastatic and invasive cancer cells. That is, similarly to the phenotype of MDA-MB-231, the phenotype was changed to CD44+CD24− or low.

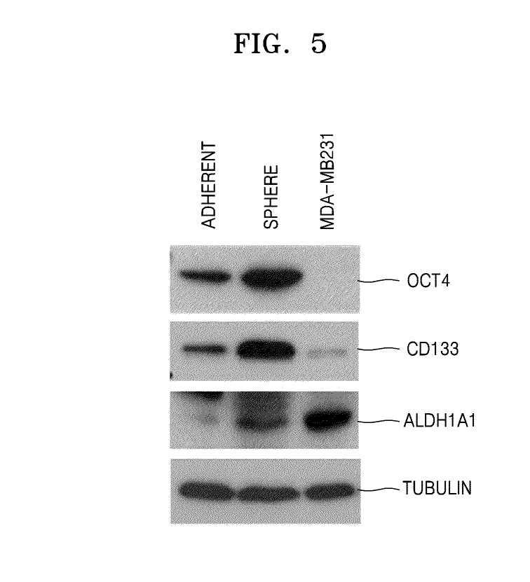

FIG. 5 illustrates results of western blotting assay of mammosphere-cultured MCF7 cells. In FIG. 5, Adherent and MDA-MB231 are as described above with reference to FIG. 4. Western blotting assay was performed using anti-Oct4, anti-CD 133, anti-ALDH1A1, and anti-Tubulin. As illustrated in FIG. 5, characteristics of cancer stem cells were obtained by inducing EMT.

According to the aforementioned results, it was confirmed that the breast cancer cell line MCF-7, lung cancer cell lines HCC827 and H1650, and prostate cancer cell lines Du145 and PC3 underwent EMT by mammosphere culture.

2. Lipidomics Analysis of EMT-Induced Cancer Cells

The EMT-induced MCF7 cells as described above were analyzed by lipidomics. Lipidomics is an analysis method of efficiently profiling various types of lipids in cells. The cells were homogenized and internal standards corresponding to various classes were introduced thereinto. The resultants were extracted using a mixture including chloroform and methanol (v:v=2:1) and separated into individual lipid species and detected using LC-MS. The lipids were identified using structural information obtained from LC-MS/MS spectrum, and the identified lipids were quantified using known concentrations of the internal standards corresponding to various classes. The obtained concentration was corrected again using the total amount of proteins contained in the cells.

As a result, in the EMT-induced MCF7 cells, the amount of poly unsaturated fatty acid (PUFA) was reduced and the amount of mono-unsaturated fatty acid (MUFA) was increased compared to MCF7 cells. Furthermore, in the EMT-induced MCF7 cells, the amount of plasmalogen having ether-linked fatty acids was significantly reduced.

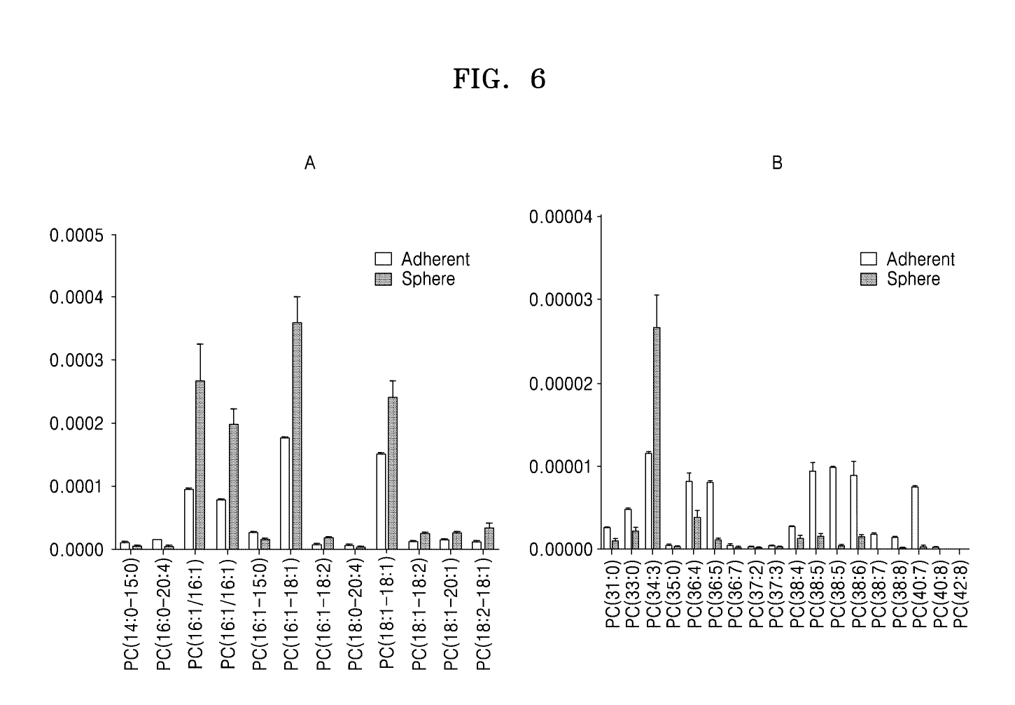

FIG. 6 illustrates analysis results of phosphatidyl choline of EMT-induced MCF7 cells using LC-MS. In the horizontal axis of FIG. 6A, regarding PC(14:0-15:0), PC(16:0-20:4), PC(16:1-15:0), etc., explaining for PC(14:0-15:0) as an example, it represents phosphatidylcholine having C14 fatty acid with no double bond and C15 fatty acid with no double bond and symbol “-” represents that whether these fatty acids attached to glycerol moiety's S1 or S2 position is not differentiated by the detection device. Regarding PC(16:1/16:1), it represents phosphatidylcholine having C16 fatty acid with one double bond at S1 position and C16 fatty acid with one double bond at S2 position and symbol “/” represents that whether these fatty acids attached to glycerol moiety's S1 or S2 position is differentiated by the detection device. In the horizontal axis of FIG. 6B, regarding PC(31:0), PC(33:0), PC(34:3), etc., explaining for PC(31:0) as an example, the number of the total carbon of fatty acid at S1 position and S2 position is 31 and the number of the total double bond(s) of fatty acid at S1 position and S2 position is 0. This type of representation is employed since the each fatty acid is not differentiated by the LC_MS. For example, if a PC has fatty acid (16:0) and fatty acid (16:0), it is represented as PC(34:0).

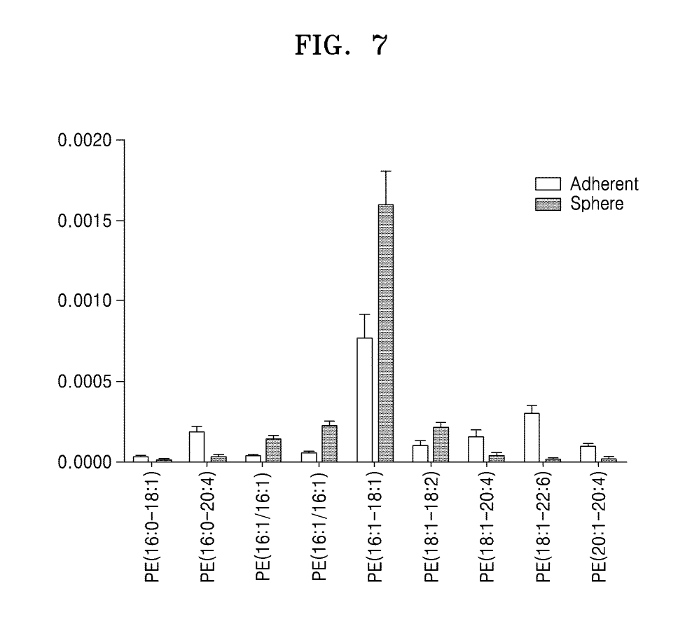

FIG. 7 illustrates analysis results of phosphatidyl ethanolamine of EMT-induced MCF7 cells using LC-MS. In the horizontal axis of FIG. 7, PE(16:0-18:1), PE(16:0-20:4), PE(16:1/16:1), etc., these representations are similarly defined as explained for FIG. 6 except that PE represents phosphatidyl ethanolamine.

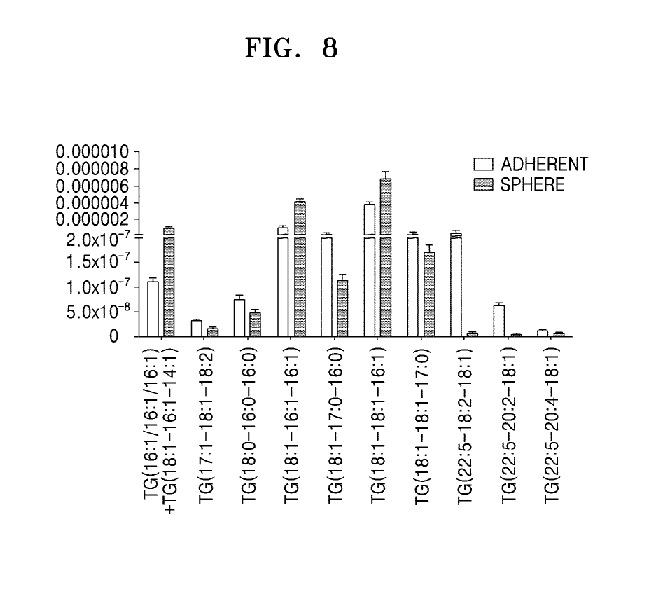

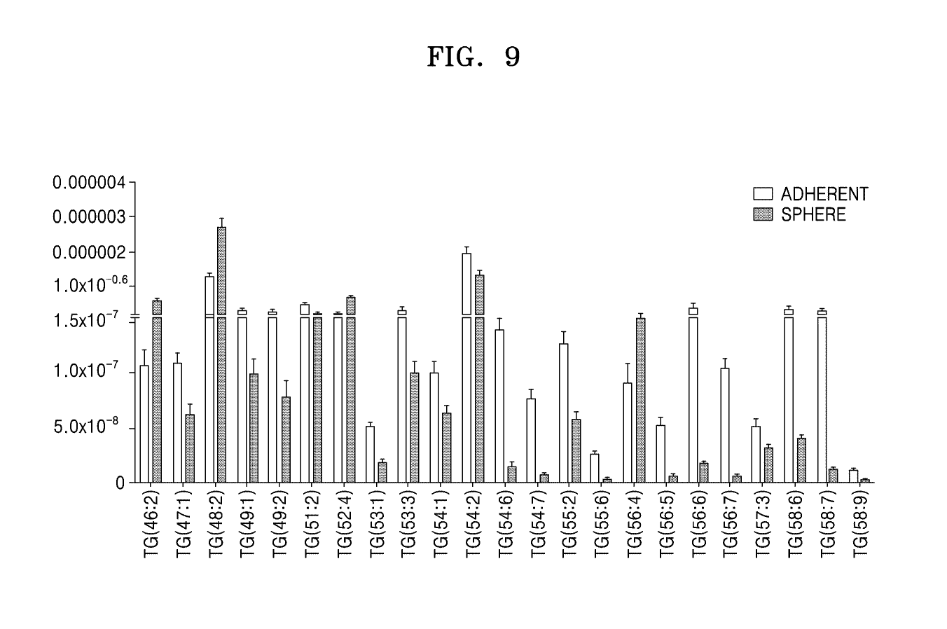

FIGS. 8 and 9 illustrate analysis results of triglyceride of EMT-induced MCF7 cells using LC-MS. In the horizontal axis of FIG. 8 and FIG. 9, TG(16:1/16:1/16:1), TG(18:1-16:1-14:1), etc. and TG(46:2), TG(47:1), etc., these representations are similarly defined as explained for FIG. 6 except that TG represents triglyceride.

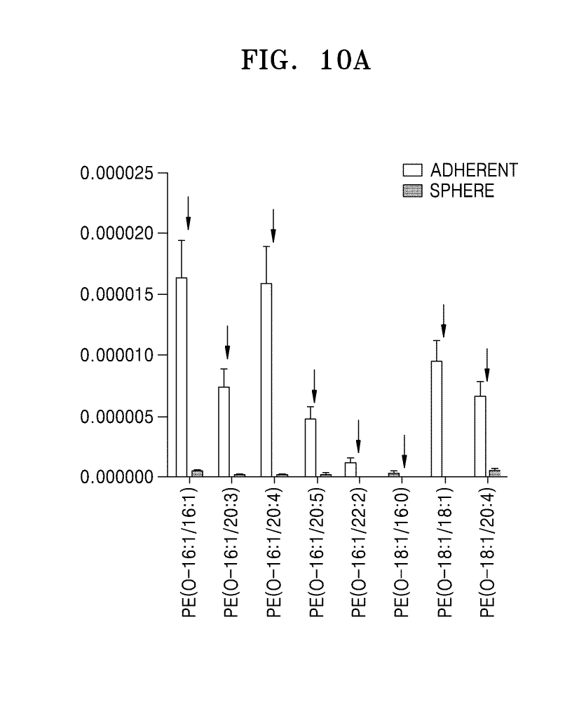

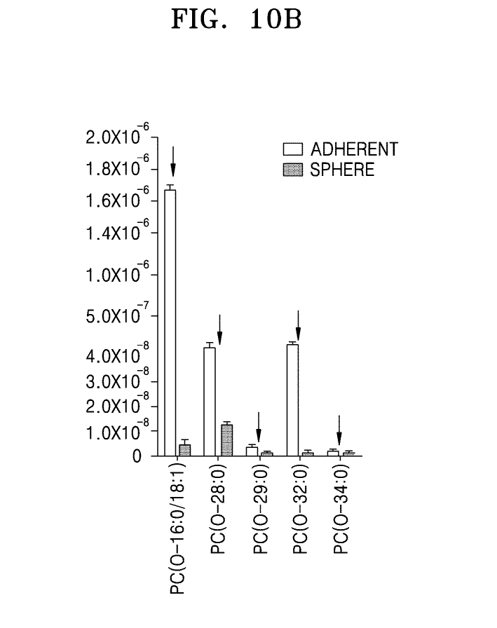

FIG. 10 illustrates analysis results of ether-linked phosphatidyl choline (a) and ether-linked phosphatidyl ethanolamine (b) of EMT-induced MCF7 cells using LC-MS. In the horizontal axis of FIG. 10, PE(O-16:1/16:1), PE(O-16:1/20:3), etc., and PC(O-16:1/18:1), PC(O-28:0), etc., these representations are similarly defined as explained for FIG. 6 except that “O-” represents “ether-linked” PE (phosphatidylethanolamine) or PC (phosphatidylcholine).

3. Identification of Expression Level of Lipid Metabolism-Related Gene of EMT-Induced Cells

In order to identify gene expression change corresponding to the change of lipids which has caused by EMT as described above in Example 1-2, a gene expression analysis was performed using a microarray on which a probe for a lipogenesis-associated gene was immobilized or RT-PCR using a primer specific to a lipogenesis-associated gene was used to analyze a gene expression profile.

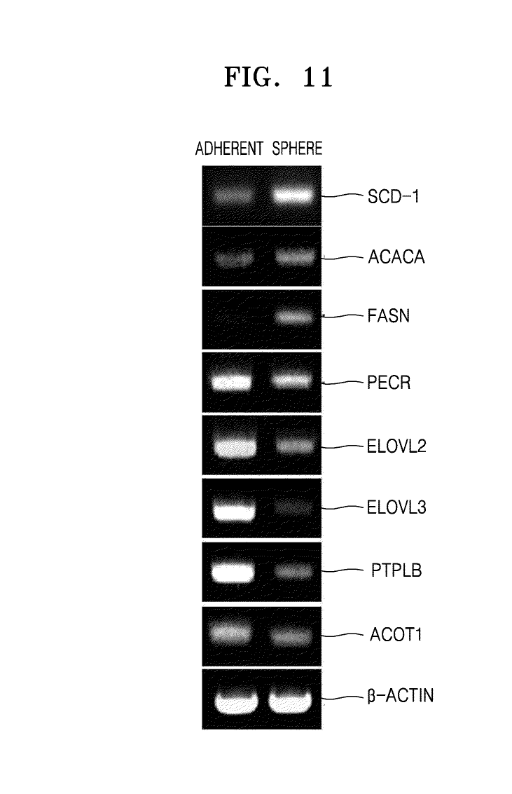

FIG. 11 illustrates analysis results of lipogenesis-associated genes in EMT-induced MCF7 cells using RT-PCR. In FIG. 11, Adherent indicates data obtained from adherent culture (control group). As shown in Table 1, expressions of mRNAs of SCD1, ACACA, and FASN were increased and expressions of PECR, ELOVL2, ELOVL3, PTPLB, and ACOT1 were reduced compared to the control group.

TABLE 1

| Gene | Fold change |
|---|---|
| SCD1 | 1.8772. |
| ACACA | 0.5317. |
| FASN | 0.9370. |
| PECR | −0.9580. |
| ELOVL2 | −0.6254. |
| ELOVL3 | −0.5959. |
| PTPLB | −1.2243. |
| ACOT1 | −0.1793. |

According to the microarray analysis, mRNA isolated from the cultured cells was reverse-transcribed and a complementary cDNA was hybridized with the microarray on which a probe is immobilized. Particularly, cRNA was prepared using 500 ng of total RNA per a sample using an IlluminaTotalPrep RNA amplification kit (AmbionInc) and hybridized with a human HT12-v4 IlluminaBeadchip gene expression array (Illumina). Fluorescent signals were scanned and analyzed using an Illumina Bead Array Reader (Illumina).

In addition, EMT was induced in breast cancer cells (MCF7), lung cancer cells (NCI-1650), and prostate cancer cells (Du145) by mammosphere culture as described above or by seeding these cells in a 60 mm dish (Nunc) containing a DMEM medium supplemented with 0.5 μM of Trichostatin A (TSA), 2.5 mM of sodium butyrate (SB), and 400 μM of CoCl₂ or RPMI-1640 (Gibco) medium to 2×10⁵ cells and culturing the cells for one day. The cultures were performed in a humidified incubator with 5% CO₂ at 37. Then, EMT-induced cancer cells were harvested, and then, western blotting and RT-PCR were performed.

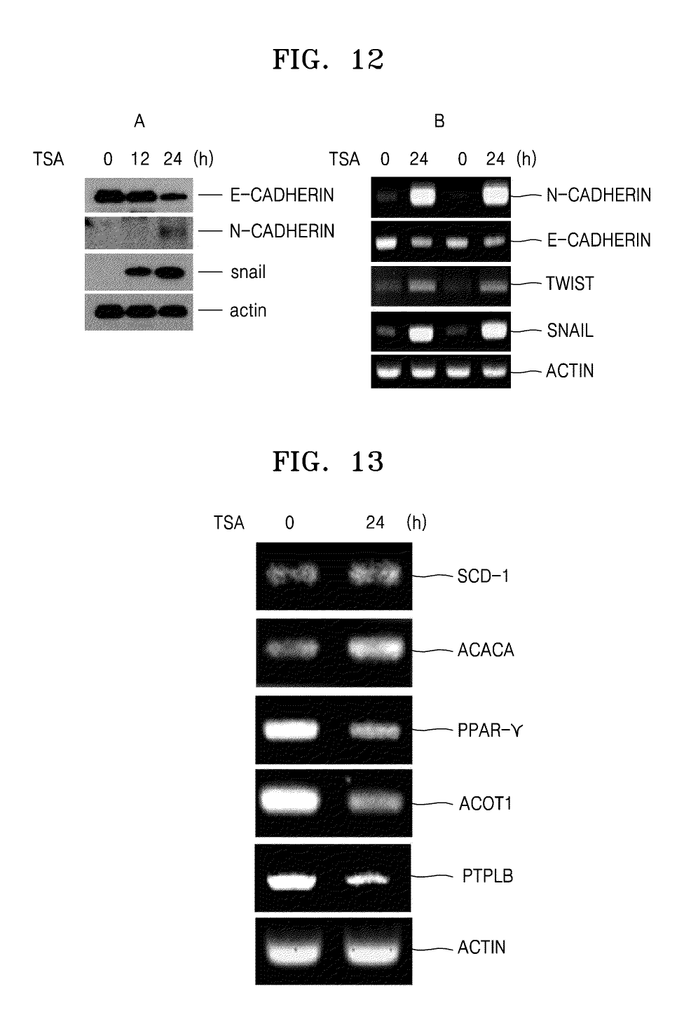

FIG. 12 illustrates western blotting results (left) and RT-PCR results (right) of EMT-induced MCF7 cells cultured in a TSA-containing medium. As illustrated in FIG. 12, the expression of E-cadherin decreased, the expressions of N-cadherein and snail gradually increased (left), and the expression of Twist increased (right).

FIG. 13 illustrates RT-PCR results of EMT-induced MCF7 cells cultured in a TSA-containing medium. As illustrated in FIG. 13, in the EMT-induced MCF7 cells by TSA, the expressions of SCD1 and ACACA increased and the expressions of ACOT1, PTPLB, and PPARγ decreased.

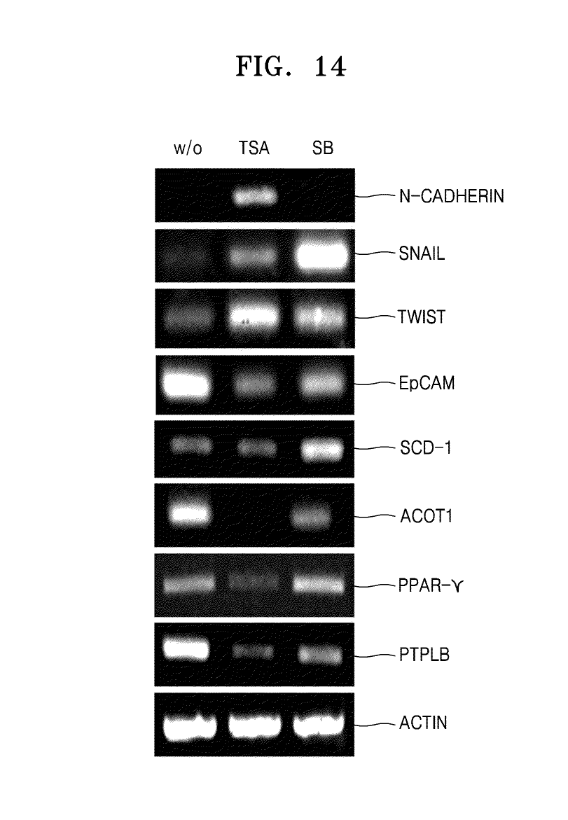

FIG. 14 illustrates RT-PCR results of EMT-induced MCF7 cells cultured in a medium including TSA or SB. As illustrated in FIG. 14, in the EMT-induced MCF7 cells by TSA, the expressions of N-cadherin, Snail, Twist, and SCD1 increased and the expressions of EpCAM, ACOT1, PTPLB, and PPARγ decreased. In the EMT-induced MCF7 cells by SB, the expressions of Snail and Twist increased and the expressions of EpCAM, ACOT1, and PTPLB decreased.

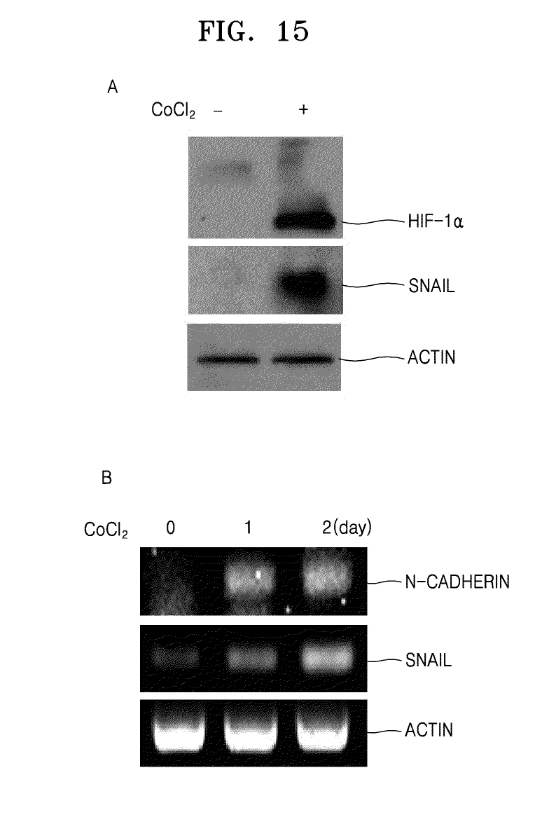

FIG. 15 illustrates western blotting results (top) and RT-PCR results (bottom) of EMT-induced MCF7 cells cultured in a CoCl₂-containing medium. As illustrated in FIG. 15, the expressions of HIF-1α, N-cadherein, and snail gradually increased.

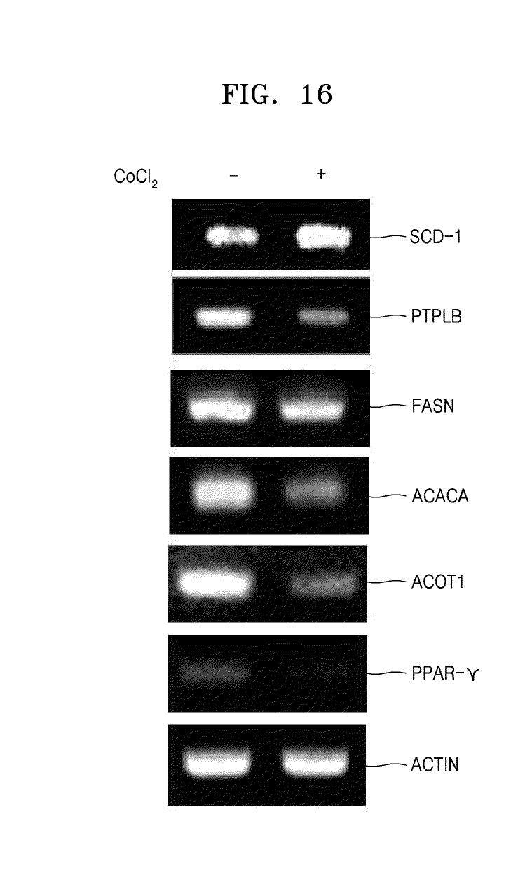

FIG. 16 illustrates RT-PCR results of EMT-induced MCF7 cells cultured in a CoCl₂-containing medium. As illustrated in FIG. 16, the expression of SCD1 increased, while the expressions of PTPLB, ACOT1, FASN, ACACA, and PPARγ decreased.

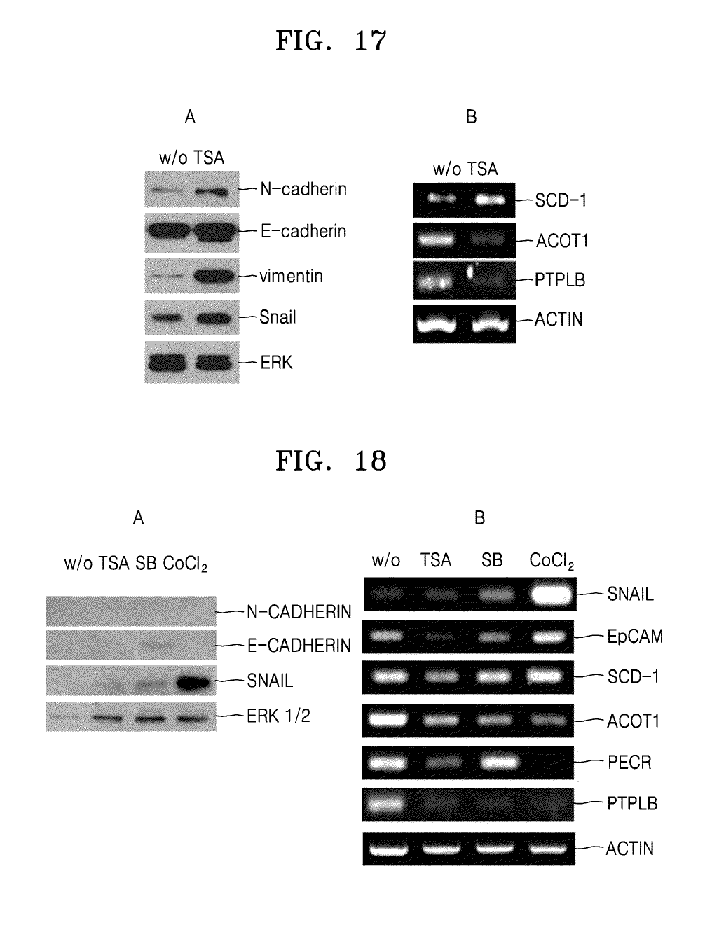

FIG. 17 illustrates western blotting results (left) and RT-PCR results (right) of EMT-induced lung cancer cell lines NCI-1650 cells cultured in a TSA-containing medium. As illustrated in FIG. 17 (left), the expressions of N-cadherein, Vimentin, and snail gradually increased. The expression of E-cadherin was similar to that of the control group. Referring to the right image of FIG. 17, the expression of SCD1 increased and the expressions of ACOT1 and PTPLB decreased.

FIG. 18 illustrates western blotting results (left) and RT-PCR results (right) of EMT-induced prostate cancer cell lines DU145 cells cultured in a medium including TSA, SB, or CoCl₂. As illustrated in FIG. 18, the expressions of SCD1, snail, and EpCAM increased and the expressions of ACOT1 and PTPLB decreased.

Primer sequences used in the RT-PCR in Example 1 are listed in Table 2 below.

TABLE 2

| Name of primer | SEQ ID NO: |
|---|---|
| ACACA-F | 1 |
| ACACA-R | 2 |
| Acot1-F | 3 |
| Acot1-R | 4 |
| β-actin-F | 5 |
| β-actin-R | 6 |
| E-cadherin-F | 7 |
| E-cadherin-R | 8 |
| ELOVL2-F | 9 |
| ELOVL2-R | 10 |
| ELOVL3-F | 11 |
| ELOVL3-R | 12 |
| EpCAM-F | 13 |
| EpCAM-R | 14 |
| FASN-F | 15 |
| FASN-R | 16 |
| Fibronectin-F | 17 |
| Fibronectin-R | 18 |
| N-cadherin-F | 19 |
| N-cadherin-R | 20 |
| PECR-F | 21 |
| PECR-R | 22 |
| PPARγ-F | 23 |
| PPARγ-R | 24 |
| PTPLB-F | 25 |
| PTPLB-R | 26 |
| SCD1-F | 27 |
| SCD1-R | 28 |
| Snail-F | 29 |
| Snail-R | 30 |
| Twist-F | 31 |
| Twist-R | 32 |
| Vimentin-F | 33 |
| Vimentin-R | 34 |

Based on the analysis results of the lipid regulatory genes obtained as described above, SCD-1, ACOT1, and PTPLB exhibited the same expression patterns among various genes. Thus, it was confirmed that these three types of genes are markers indicating general EMT-undergoing cancer cells regardless of the origin or type of cancer.

These three genes are considered to participate in lipid metabolism as follows, but the scope of the invention is not limited thereto. First, SCD1 serves to introduce a double bond into saturated fatty acid to produce mono-unsaturated fatty acid. Thus, the increase in SCD-1 may result in an increase in MUFA. Second, PTPLB plays an important role in synthesis of PUFA in endoplasmic reticulum. Thus, the reduction in PTPLB may result in a reduction in PUFA. Third, ACOT1 serves to convert free fatty acid into fatty acyl-CoA. Free fatty acid is converted into lipid having esterified fatty acid such as phosphatidylcholine/triglyderide after forming fatty acyl-CoA. Thus, the reduction in ACOT1 may induce an increase in free fatty acid and a reduction in esterified fatty acid.

In conclusion, SCD-1 may induce an increase in MUFA, PTPLB may induce a reduction in PUFA, and ACOT1 may induce an increase in free fatty acid.

As described above, according to the method of obtaining information for identifying EMT-undergoing tumor cells in a sample according to the one or more of the above embodiments of the present disclosure, information for identifying EMT-undergoing tumor cells in the sample may be efficiently obtained.

As described above, according to the method of identifying EMT-undergoing tumor cells in a sample according to the one or more of the above embodiments of the present disclosure, EMT-undergoing tumor cells in the sample may be efficiently identified.

As described above, according to the method of diagnosing a subject having a tumor according to the one or more of the above embodiments of the present disclosure, a subject having a tumor may be efficiently diagnosed.

As described above, according to the composition or kit for identifying EMT-undergoing tumor cells in a sample according to the one or more of the above embodiments of the present disclosure, EMT-undergoing tumor cells in the sample may be efficiently identified.

It should be understood that the exemplary embodiments described therein should be considered in a descriptive sense only and not for purposes of limitation. Descriptions of features or aspects within each embodiment should typically be considered as available for other similar features or aspects in other embodiments.

It should be understood that the exemplary embodiments described herein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of obtaining information for identifying tumor cells undergoing epithelial-mesenchymal transition (EMT) in a sample, the method comprising
providing a sample comprising a tumor cell obtained from a subject;
measuring in the sample an expression level of at least one selected from the group consisting of stearoyl-CoA desaturase1 (SCD1), acyl-CoA thioesterase (ACOT1), and protein tyrosine phosphatase-like member B (proline instead of catalytic arginine) (PTPLB); and
comparing the expression level with that of a control sample.

2. The method of claim 1, wherein the sample comprises a tumor tissue, blood, bone marrow, lymph, saliva, tears, urine, mucus, amniotic fluid, or any combination thereof.

3. The method of claim 1, wherein the control sample comprises normal cells, tumor cells that have not undergone EMT, or any combination thereof; or tumor cells known to have undergone EMT.

4. The method of claim 1, wherein the cells of the sample comprise circulating tumor cells (CTCs), breast cancer cells, prostate cancer cells, lung cancer cells, colorectal cancer cells, gastric cancer cells, ovarian cancer cells, endometrial cancer cells, liver cancer cells, esophageal cancer cells, pancreatic cancer cells, or thyroid cancer cells.

5. The method of claim 4, further comprising measuring in the sample an expression level of at least one selected from the group consisting of acetyl-CoA carboxylase alpha (ACACA), fatty acid synthase (FASN), peroxisomal trans-2-enoyl-CoA reductase (PECR), elongation of very long chain fatty acids (FEN1/Elo2, SUR4/Elo3, yeast)-like 2 (ELOVL2), elongation of very long chain fatty acids (FEN1/Elo2, SUR4/Elo3, yeast)-like 3 (ELOVL3), and peroxisome proliferator activator receptor-gamma (PPARγ); and comparing the expression level with that of a control sample.

6. A method of identifying tumor cells as undergoing or having undergone epithelial-mesenchymal transition in a sample, the method comprising:
providing a sample comprising a tumor cell obtained from a subject;
measuring an expression level of at least one selected from the group consisting of stearoyl-CoA desaturase1 (SCD1), acyl-CoA thioesterase (ACOT1), and protein tyrosine phosphatase-like member B (proline instead of catalytic arginine) (PTPLB) in the sample;
comparing the expression level with that of a control sample; and
identifying the tumor cells as undergoing EMT or having undergone EMT when at least one selected from the group consisting of a higher expression level of SCD1, a lower expression level of ACOT1, and a lower expression level of PTPLB, as compared to the control sample, is observed in the tumor cells,
or identifying the tumor cells as not undergoing or not having undergone EMT when at least one selected from the group consisting of a lower expression level of SCD1, a higher expression level of ACOT1, and a higher expression level of PTPLB, as compared to the control sample is observed in the tumor cells,
wherein the control sample is a normal cell or tumor cell that has not undergone EMT.

7. The method of claim 6, wherein the sample comprises a tumor tissue, blood, bone marrow, lymph, saliva, tears, urine, mucus, amniotic fluid, or any combination thereof.

8. The method of claim 6, wherein the control sample comprises normal cells, tumor cells that have not undergone EMT, or tumor cells that have undergone EMT.

9. The method of claim 6, wherein the cells of the sample comprise circulating tumor cells (CTCs), breast cancer cells, prostate cancer cells, lung cancer cells, colorectal cancer cells, gastric cancer cells, ovarian cancer cells, endometrial cancer cells, liver cancer cells, esophageal cancer cells, pancreatic cancer cells, or thyroid cancer cells.

10. The method of claim 6, further comprising:
measuring in the sample an expression level of at least one selected from the group consisting of acetyl-CoA carboxylase alpha (ACACA), fatty acid synthase (FASN), peroxisomal trans-2-enoyl-CoA reductase (PECR), elongation of very long chain fatty acids (FEN1/Elo2, SUR4/Elo3, yeast)-like 2 (ELOVL2), elongation of very long chain fatty acids (FEN1/Elo2, SUR4/Elo3, yeast)-like 3 (ELOVL3), and peroxisome proliferator activator receptor-gamma (PPARγ); and
comparing the expression level with that of a control sample;
determining that the subject has cancer cells undergoing or having undergone epithelial-mesenchymal transition when at least one selected from the group consisting of a higher expression level of ACACA, a higher expression level of FASN, a lower expression level of PECR, a lower expression level of ELOVR2, a lower expression level of ELOVR2, and a lower expression level of PPARγ as compared to a control sample is observed in the tumor cells,
or determining that the subject does not have tumor cells undergoing or having undergone EMT when at least one selected from the group consisting of a lower expression level of ACACA, a lower expression level of FASN, a higher expression level of PECR, a higher expression level of ELOVR2, a higher expression level of ELOVR2, and a higher expression level of PPARγ compared to a control sample is observed in the tumor cells;
wherein the control sample is a normal cell or tumor cell that has not undergone EMT.

11. A composition for identifying tumor cells undergoing EMT or having undergone EMT in a sample, the composition comprising a reagent to determine an expression level of at least one selected from the group consisting of stearoyl-CoA desaturase1 (SCD1), acyl-CoA thioesterase (ACOT1), and protein tyrosine phosphatase-like member B (proline instead of catalytic arginine) (PTPLB).

12. The composition of claim 11, wherein the reagent comprises a substance capable of binding to at least one of the SCD1, ACOT1, and PTPLB or capable of amplifying nucleotides encoding at least one of the SCD1, ACOT1, and PTPLB.

13. The composition of claim 12, wherein the substance is an antibody or an antigen-binding fragment thereof, and the nucleotides are mRNA.

14. The composition of claim 11, further comprising a reagent to determine an expression level of at least one of the ACACA, FASN, PECR, ELOVL2, ELOVL3, and PPARγ.

15. A method of diagnosing a subject having a tumor undergoing or having undergone EMT, the method comprising:
providing a sample comprising a tumor cell obtained from a subject;
measuring an expression level of at least one selected from the group consisting of stearoyl-CoA desaturase1 (SCD1), acyl-CoA thioesterase (ACOT1), and protein tyrosine phosphatase-like member B (proline instead of catalytic arginine) (PTPLB) in the sample;
comparing the expression level with that of a control sample; and
diagnosing the subject as having tumor cells undergoing EMT or which have undergone EMT based upon the comparison of the expression level with that of a control sample when at least one selected from the group consisting of a higher expression level of SCD1, a lower expression level of ACOT1, and a lower expression level of PTPLB, as compared to a control sample is observed in the tumor cells,
wherein the control sample is a normal cell, or tumor cell that has not undergone EMT.

16. The method of claim 15, wherein the control sample comprises normal cells, tumor cells that have not undergone EMT, or tumor cells that have undergone EMT.

17. The method of claim 15, wherein the cells of the sample comprise circulating tumor cells (CTCs), breast cancer cells, prostate cancer cells, lung cancer cells, colorectal cancer cells, gastric cancer cells, ovarian cancer cells, endometrial cancer cells, liver cancer cells, esophageal cancer cells, pancreatic cancer cells, or thyroid cancer cells.

18. The method of claim 15, further comprising:
measuring an expression level of at least one selected from the group consisting of acetyl-CoA carboxylase alpha (ACACA), fatty acid synthase (FASN), peroxisomal trans-2-enoyl-CoA reductase (PECR), elongation of very long chain fatty acids (FEN1/Elo2, SUR4/Elo3, yeast)-like 2 (ELOVL2), elongation of very long chain fatty acids (FEN1/Elo2, SUR4/Elo3, yeast)-like 3 (ELOVL3), and peroxisome proliferator activator receptor-gamma (PPARγ) in the sample;
comparing the expression level with that of the control sample; and
determining that the subject has cancer cells undergoing or having undergone epithelial-mesenchymal transition when at least one selected from the group consisting of a higher expression level of ACACA, a higher expression level of FASN, a lower expression level of PECR, a lower expression level of ELOVR2, a lower expression level of ELOVR3, and a lower expression level of PPARγ as compared to a control sample is observed in the tumor cells,
or determining that the subject does not have cancer cells undergoing or having undergone epithelial-mesenchymal transition when at least one selected from the group consisting of a lower expression level of ACACA, a lower expression level of FASN, a higher expression level of PECR, a higher expression level of ELOVR2, a higher expression level of ELOVR3, and a higher expression level of PPARγ as compared to a control sample is observed in the tumor cells,
wherein the control sample is a normal cell, or tumor cell that has not undergone EMT.
